(19) United States
(12) Patent Application Publication
(10) Pub. No.: US 2024/0165174 A1
(43) Pub. Date: May 23, 2024

(54) SPIRULINA-BASED COMPOSITION AND USES THEREOF FOR REINFORCING IMMUNITY

(71) Applicant: Axeen Pharma, Monaco (MC)

(72) Inventor: Sossio Morra, Monaco (MC)

(21) Appl. No.: 18/283,424

(22) PCT Filed: Mar. 29, 2022
(86) PCT No.: PCT/EP2022/058236

§ 371 Date: Sep. 21, 2023

(30) Foreign Application Priority Data
Mar. 30, 2021 (FR) 2103289

(51) Int. Cl. A61K 35/748; A61K 9/00; A61K 9/48; A61K 31/355; A61K 31/4415; A61K 31/593; A61K 33/04; A61K 36/15; A61K 36/899; A61P 31/14

(52) U.S. Cl. CPC A61K 35/748; A61K 9/0053; A61K 9/4816; A61K 9/4858; A61K 9/4866; A61K 31/355; A61K 31/4415; A61K 31/593; A61K 33/04; A61K 36/15; A61K 36/899; A61P 31/14

(57) ABSTRACT

The invention relates to a composition comprising spirulina. The invention is characterized in that the composition comprises an aqueous extract of pollen cytoplasm, said pollen(s) being obtained from plants belonging to the Secale, Zea, Pinus and/or Dactylis genera, and optionally: an extract of pistil(s); selenium; and/or vitamins.

### TECHNICAL FIELD

The invention relates to an oral composition for human use comprising spirulina for use in immune reinforcement.

More particularly, it covers such a composition comprising other ingredients, for use as a medicament or dietary supplement and in particular as an immunomodulator.

### PRIOR ART

Immunity is an organism's ability to defend itself against foreign substances and pathogens. The immune system is the body's set of defense mechanisms. It is made up of organs, tissues, cells and molecules that ensure an organism's immunity.

The term “immune response” refers to the triggering of the immune system in the face of an aggressor or disease. In this way, the immune system ensures the organism's integrity against external elements, as well as against constitutive but abnormal elements, such as tumor cells. It helps contain the development and dissemination of pathogens in the body, enabling their elimination.

The external pathogens the immune system has to contend with are mainly viruses, parasites, yeasts, fungi and bacteria.

The immune response triggered by a pathogen is characterized by two functional aspects: Natural immunity, on the one hand, which is innate and non-specific, and specific immunity, on the other hand, which is acquired and adaptive.

Natural immunity is the first line of defense. It is identical in all individuals of the same species, whatever the pathogen, and is present from birth.

Specific immunity is said to be specific because the associated immune response is directed against a single antigen: once pathogens have succeeded in breaking through the body's first defense barrier (the inflammatory response), a second line of defense comes into action.

An immune reaction will lead to the differentiation of immune cells and the production of numerous effectors, which may be cellular, such as macrophages and monocytes, or molecular, such as cytokines, interleukins or antibodies.

Molecular effectors thus enable the organism to fight the pathogen.

More specifically, the first step is recognizing pathogens with foreign patterns by phagocytic cell receptors (cellular effectors). This triggers the release of chemical mediators of inflammation (interleukins, histamine, TNF) by the phagocytic cells. Interleukins and cytokines mediate cell-to-cell communication and thus facilitate the arrival of other cells at the site of infection. They trigger the inflammatory response necessary for immune reactions. They also enable phagocytosis of the pathogen. Next, the defense reaction is amplified, with T-helper lymphocytes attracting the various host defenses to the site where the pathogen has been localized. Finally, the pathogen is eliminated: B lymphocytes produce antibodies that bind to foreign antigens, cytotoxic T lymphocytes release destructive enzymes, and macrophages phagocytose infected cells.

Every day, the immune system is put to the test, so an active immune defense is essential.

Since ancient times, diseases have decimated entire populations in the space of a few months or even a few days, triggering terror among the inhabitants in the face of an unknown evil. Pandemics caused by bacteria or viruses have been known since ancient times: for example, the plague of Athens, the first documented pandemic in history, decimated a third of the city of Athens in 430 BC. The Antonine Plague, the Black Death, the Spanish flu, cholera, Asian flu and AIDS are just some of the epidemics that have left their mark on history.

More recently, other viruses have emerged. Examples include the SARS, MERS-Cov, Ebola, Zika and H1N1 epidemics. Finally, the Covid-19 pandemic refers to “Coronavirus Disease 2019”, the disease caused by a virus in the Coronaviridae family, SARS-CoV-2. This infectious disease is a zoonosis, the origin of which is still being debated, that emerged in December 2019 in the city of Wuhan, in China's Hubei province.

Viruses are microscopic infectious particles that can only replicate by entering cells and using their cellular machinery.

When viruses enter the body, immune cells recognize the pathogen as foreign and activate an immune response to induce immune cell proliferation and the production of molecular effectors to eliminate the virus from the body.

It's a well-known fact that with age or illness, immunity weakens and the body becomes more vulnerable to infection.

A balanced diet and a healthy lifestyle also help maintain a good immune system. In fact, 70% of the immune system is located in the intestine. This organ, and more specifically the bacteria it contains, that is the microbiota, play a major role in defending against pathogens. Preserving the integrity of the intestinal microbiota is essential for maintaining good defenses, and probiotics can help.

Immune-boosting elements include vitamins, trace elements, probiotics, minerals, cofactors and more. They are often found either in the diet, or combined in the form of food supplements.

Vitamins are also known for their contribution to the proper functioning of the immune system. A well-balanced diet helps to absorb vitamins. Vitamins are readily available in the form of food supplements.

Other plants and organisms have also been studied and recognized for their effectiveness on the immune system. These include blueberries, cranberries, goji berries, strawberries, chili peppers, garlic, ginger, chia seeds, flax, quinoa, cocoa, mace and spirulina.

In recent years, studies have shown that spirulina stimulates a large number of physiological mechanisms and boosts the immune system. This cyanobacterium is rich in vitamins, minerals, proteins, linolenic acid and antioxidants. The paper entitled “Impact of daily supplementation of Spirulina platensis on the immune system of naïve HIV-1 patients in Cameroon: A 12-months single blind, randomized, multicenter trial”, Ngo-Matip et al. (2015). Nutrition Journal. showed that spirulina effectively delays the progression of HIV by improving immune responses.

Today, there are many dietary supplements on the market containing spirulina in tablet, capsule and powder form. Most of these supplements use spirulina powder without an extraction process, containing a high percentage of phycocyanin.

These include products from the NUTRIMEA™, NUTRI&CO™ and NUTRALIE™ laboratories. They are advertised as improving endurance and recovery after exercise, boosting vitality, eliminating nutritional deficiencies, combating fatigue, helping to control weight, and strengthening the immune system and natural defenses. These food supplements come in tablet or capsule form. They contain spirulina alone, or spirulina combined with vitamins and chlorophyll.

Patent CN108851055 describes a food composition used as a remedy for intestinal pain. The composition described includes spirulina and pollen.

The paper “Photosynthetically Controlled Spirulina, but Not Solar Spirulina, Inhibits TNF-αSecretion: Potential Implications for COVID19 Related Cytokine Storm Therapy” Tzachor, A. et Al. (2021) Mar Biotechnol 23, 149-155 discloses the effect of photosynthetically-controlled spirulina (Arthrospira platensis), called LED Spirulina, on macrophages and monocytes activated by toll-like receptor-binding LPS (lipopolysaccharides). LED spirulina has also been shown to reduce TNF-α secretion induced by macrophages and monocytes. In this case, spirulina has an anti-inflammatory effect when the level of inflammatory cytokines is high.

Spirulina is thus able to modify the body's immune response, in what is known as an immunomodulator. Depending on how it is used, it will either boost the immune system (basal state of cytokine levels) or have an anti-inflammatory effect (high cytokine levels).

Many viral infections, including the Covid-19 or SARS-Cov-2 coronavirus, trigger hemophagocytosis syndrome (macrophagic activation syndrome). This abnormality results from cytokine deregulation (cytokine shock), leading to overexpression of inflammatory cytokines, notably TNF-α.

TNF-α is an inflammatory cytokine secreted by activated macrophages. In the case of SARS-Cov-2, it proves to be responsible for severe forms, and thus for severe acute respiratory syndrome.

### Technical Problem

In view of the foregoing, one problem which the present invention sets out to solve is to implement an oral composition capable of increasing immunomodulation, and thus boosting the immune system in humans in an effective way to cope with, for example, emerging viruses such as SARS-Cov-2 or other infections.

Thus, the Applicant unexpectedly discovered that a composition comprising spirulina, on the one hand, and pollen mainly obtained from plants belonging to the Pinaceae and/or Poaceae family, on the other, could boost the immune system in humans.

Such a composition taken alone and, advantageously, in association with a plurality of other ingredients acting in synergy, exhibits remarkable immune system potentiation activity, particularly in activating cells of the body's first line of defense for the treatment and/or prevention of bacterial/viral, fungal or parasitic infections.

### Technical Solution

The invention's solution to this technical problem has as its first object an oral composition comprising spirulina or a spirulina extract, characterized in that it further comprises an aqueous extract of pollen cytoplasm, said pollen(s) being obtained from plants belonging to the Secale, Zea, Pinus and/or Dactylis genera, and optionally:
extract of pistil(s);
selenium; and/or
vitamins.

It also relates to an oral composition comprising spirulina or a spirulina extract, characterized in that it further comprises an aqueous extract of pollen cytoplasm, said pollen(s) being obtained from plants belonging to the Secale, Zea, Pinus and/or Dactylis genera, and optionally:
extract of pistil(s);
selenium; and/or
vitamins;
   for use as a medicine or dietary supplement.

It also relates to an oral composition comprising spirulina or a spirulina extract and pollen obtained from plants belonging to the Secale, Zea, Pinus and/or Dactylis genera, for use as an immunomodulator.

It also relates to a method for preparing a composition according to the invention, comprising the following steps of:
a. aqueous extraction of pollen cytoplasm and optionally pistil cytoplasm;
b. spray-drying the aqueous extract(s) thus obtained;
c. recovering said aqueous extracts and mixing with spirulina or spirulina extract and optionally:
selenium; and/or
vitamins; and
d. recovering the resulting composition.

### Benefits Provided

In particular, the Applicant has been able to develop a composition that is particularly effective for use in activating and strengthening the immune system.

### BRIEF DESCRIPTION OF THE DRAWINGS

The invention and its advantages will be better understood on reading the following description and non-limiting embodiments, shown with reference to the appended drawings wherein:

FIG. 1 shows the procedure used to generate results for studying the effect of composition A on cytokine production, as detailed in example 3.

FIG. 2 shows the effect of composition A on the production of various cytokines in human monocytes, as well as the concentrations of composition A effective for significant cytokine production as detailed in example 4.

FIG. 3 shows IL-1p production by different compositions comprising spirulina, pollen extracts, selenium, vitamin B6 and/or vitamin D3, as detailed in example 5.

FIG. 4 shows TNF-α production by different compositions comprising spirulina, pollen extracts, selenium, vitamin B6 and/or vitamin D3, as detailed in example 5.

FIG. 5 shows IL-6 production by different compositions comprising spirulina, pollen extracts, selenium, vitamin B6 and/or vitamin D3, as detailed in example 5.

FIG. 6 shows MCP-1 production by different compositions comprising spirulina, pollen extracts, selenium, vitamin B6 and/or vitamin D3, as detailed in example 5.

FIG. 7 shows IL-8 production by different compositions comprising spirulina, pollen extracts, selenium, vitamin B6 and/or vitamin D3, as detailed in example 5.

FIG. 8 shows PGE-2 production by different compositions comprising spirulina, pollen extracts, selenium, vitamin B6 and/or vitamin D3, as detailed in example 5.

FIG. 9 shows the effect of composition A on the inhibition of the RBD (of the Spike protein) at the ACE2 receptor as detailed in example 6.

### DESCRIPTION OF EMBODIMENTS

In this description, unless otherwise specified, it is understood that, when an interval is given, it includes the upper and lower bounds of said interval.

The invention concerns an oral composition comprising spirulina.

The composition according to the invention can be administered orally, in one or more identical or different formulations. It is available in any dosage form normally used for oral administration, including gelcaps, tablets, capsules, softgels, lozenges, sachets, tubes, vials, chewing gum, pearls, emulsions, suspensions, liquids, solutions, ampoules, beverages, syrups, powders, solids, soft gels and semi-solids.

Advantageously, the composition is in the form of a tablet, gelcap, capsule, semi-solid, solid, liquid, or powder.

Said composition comprises spirulina or spirulina extract. Spirulina is a product made from cyanobacteria of the Arthrospira genus.

These are blue microscopic photosynthetic bacteria, usually dried and ground.

The most frequently marketed species is Arthrospira platensis, grown mainly in China, the United States, France and Africa. To a lesser extent, commercially available spirulina may also be derived from Arthrospira maxima (syn. Spirulina maxima).

Spirulina is marketed in several forms:
industrial methods: tablets, powder, capsules and liquid;
traditional methods: sticks, flakes and micro-needles (unprocessed, edible version);
and sometimes in the form of processed food products, including homemade pasta.

According to the invention, the spirulina used in the compositions is an extract of Arthrospira platensis (syn. Spirulina platensis). Preferably, polysaccharides extracted from Spirulina platensis are used in the composition according to the invention, more preferentially an aqueous extract of lipopolysaccharides from Spirulina platensis.

According to a particular embodiment of the invention, the spirulina extract used can be a crude spirulina extract prepared by extracting freeze-dried material with ethanol. This crude extract is prepared, for example, using the method described in patent EP1301191.

The composition according to the invention further comprises at least pollen, and optionally pistil, obtained from plants belonging to the Poaceae and/or Pinaceae families.

The grasses (Poaceae), also called Gramineae (Gramineae), are a family of monocotyledonous plants of the order of the Poales. This family, composed of about 12,000 species grouped together in 780 genera, comprises most species commonly called “herbs” and “cereals”. They are generally herbaceous plants, more rarely ligneous (bamboos).

Like all anemophilic pollens, the Poaceae pollen is of spherical or slightly ellipsoidal shape with reduced ornaments. The single aperture (or pore) is round: this is one of the criteria of the monocotyledons. The pollen of the poaceae is small and light. The size is of the order of 40 microns. For cereals, the size is 60 to 100 microns.

The pinaceous family (Pinaceae), or conifers, groups together gymnosperm plants; it comprises 220-250 species divided into 11 genera. These are trees or shrubs, from temperate regions, either with needle or scale leaves that are persistent, or shed annually like those of larches. In this family, the indigenous species in France are among the genera Abies (fir trees), Picea (spruce), Larix (European larch), Pinus (pines).

Pinaceae produce large pollen grains in an abundant manner, the size of which is generally between 40 and 100 microns. They do not have pores. The pollen grains of pines, fir trees, spruce and cedar have two sacci which facilitate their suspension in air. The pollen grains from larches and firs are more or less spherical and do not have sacci.

Preferentially, the plants from which pollen and optionally pistils are obtained are selected from the genera Secale, Zea, Pinus and/or Dactylis, or a mixture thereof.

More particularly, the plants from which the pollen and optionally pistil are obtained are selected from the Secale cereale L. (rye), Zea mays L. (corn), Pinus sylvestris L. (pine), and/or Dactylis glomerata L. (orchard grass) species, or a mixture thereof.

Preferably, the plants from which the pollen is collected are freshly harvested. The pollen used for the present invention may be a pollen harvested by insects (such as bee pollen) or collected by human action. Bee pollen for example contains pollen, but also nectar and bee saliva. The pollen harvested via human intervention is free of such additional ingredients. Preferably, said pollens for the present compositions are obtained only by human intervention. This again makes it possible to standardize the final product.

The compositions used according to the present invention are advantageously rich in superoxide dismutase (SOD), tannins, polyphenols, vitamins, enzymes and trace elements, amino acids, fatty acids and minerals. Furthermore, the compositions used according to the present invention do not contain hormones, such as phytoestrogens.

Advantageously, cytoplasmic pollen extracts are obtained from the cytoplasm (inside part of the pollen seed without its envelope). Since the envelope is generally a source of allergens, and an obstacle to the availability of the compounds of the cytoplasm, the use of a cytoplasmic pollen extract has an obvious advantage compared to the use of a natural pollen extract. Such purified cytoplasmic extracts of specific, standardized pollen also have a high content of superoxide dismutase (SOD), tannins, polyphenols, vitamins, enzymes and trace elements, amino acids, fatty acids and minerals, as well as beneficial proteins and carbohydrates. The concentration of beneficial compounds in the different extracts is considerably greater than the amount of interesting compounds in pollens in the raw state.

The pollen, and optionally the pistil, used in the compositions according to the invention is advantageously a cytoplasmic extract of pollen and/or pistil(s), preferably selected from the species Secale cereale L. (rye), Zea mays L. (corn), Pinus sylvestris L. (pine), and/or Dactylis glomerata L. (orchard grass), or a mixture thereof.

The pollen and optionally pistil extracts according to the invention may be oily and/or aqueous extracts.

According to the invention, the term extraction is used when a solvent is used, advantageously environmentally friendly, such as water, glycerin, glycols, ethers, oils, hydroalcoholic mixtures, ethanol and other alcohols, on a plant raw material to extract certain compounds or molecules therefrom, after a possible mixing, decanting and filtration.

The solvent may then be partially or totally eliminated to obtain an extract.

Oily extract is understood to mean an extract containing fat-soluble active ingredients obtained by extraction, for example maceration, infusion, digestion, decoction, percolation or even leaching, preferentially maceration at ambient temperature of between 15 and 27° C., of a plant raw material in an oily solvent, such as an ether, a ketone or an oil.

By way of non-limiting examples, the oily solvent is an ether such as diethyl ether or a ketone such as acetone.

The composition used according to the invention preferentially comprises an aqueous extract of pollen, and optionally an aqueous extract of pistil(s).

Aqueous extract is understood to mean an extract containing water-soluble active ingredients obtained by extraction, for example a hydrodistillation, maceration, infusion, digestion, decoction, percolation or even leaching, preferentially maceration at low temperature comprised between 15 and 40° C., of a plant raw material in an aqueous solvent, that is a solvent comprising water taken alone or advantageously mixed with other solvent(s) such as an alcohol, a ketone, and/or a nonionic surfactant.

By way of non-limiting examples, the aqueous solvent is chosen from a mixture comprising predominantly water in combination with an alcohol such as ethanol, a ketone such as acetone, and/or a nonionic surfactant.

The pollen extracts, and optionally pistil extracts, used in the composition according to the invention are preferentially aqueous extracts of pollen and/or pistil cytoplasm.

Even more preferentially, the pollen, and optionally the pistil, used in the compositions according to the invention is advantageously an aqueous extract of cytoplasm from pollen and/or pistil(s), preferably selected from the species Secale cereale L. (rye), Zea mays L. (corn), Pinus sylvestris L. (pine), and/or Dactylis glomerata L. (orchard grass), or a mixture thereof.

Preferably, the composition according to the invention comprises an aqueous extract of pistil cytoplasm obtained from the Zea plant.

In addition to spirulina and pollen, the composition according to the invention advantageously also comprises pistil as described above, a trace element which is preferentially selenium, and/or vitamins.

Essential trace elements are inorganic elements whose inadequate intake can be expected to cause biological disturbances and loss of vitality. They are present in the human body in very small quantities (<0.01% of body weight), but in very constant concentrations. Chemically, they are metalloids with a variable number of valencies, whose properties and digestive absorption will vary according to valency. Twelve elements are essential, including chromium, copper, iron, iodine, selenium and zinc.

Preferably, the composition as described above additionally comprises selenium. Selenium (symbol Se in the Periodic Table of the Elements) is bound to various proteins and mainly stored in muscles.

Selenium is important for the functioning of selenoproteins, such as glutathione peroxidase and thioredoxin reductase, which act as redox regulators and cellular antioxidants, potentially countering the free radical species produced during oxidative stress.

These enzymes help neutralize the excess of free radicals in the body, which accelerate cellular aging and promote the onset of various diseases.

Preferably, the composition as described above also comprises at least one vitamin.

Vitamins are organic substances essential to the body. The human body generally cannot produce them on its own, so their dietary intake is essential.

Vitamins help to build the body, keep it functioning and maintain it.

Among the vitamins that can be used in the composition according to the invention are:
vitamin A,
the eight group B vitamins, that is vitamins B1, B2, B3, B5, B6, B8, B9, B12,
vitamin C
vitamin D in D2 and/or D3 form,
vitamin E, and
vitamin K.

More preferentially, the vitamins usable in the composition according to the invention are chosen from vitamin D3, B6 or E, taken alone or as a mixture.

Vitamin D3 is a form of vitamin D, also known as cholecalciferol. Vitamin D has a dual origin: it is supplied by the diet and synthesized by the skin under the action of the sun's UVB rays. The essential function of vitamin D is to increase the intestine's capacity to absorb calcium and phosphorus, thereby:
ensuring optimal mineralization of mineralized tissues, in particular bone, cartilage and teeth;
helping maintain plasma calcium and phosphorus levels.

Vitamin B6 is a water-soluble vitamin represented by three main forms: pyridoxine, pyridoxal and pyridoxamine.

Found in a wide variety of plant and animal foods, vitamin B6 is essential for healthy cell function, particularly in the nervous and skin systems.

Vitamin E is a fat-soluble vitamin covering a group of eight organic molecules, four tocopherols and four tocotrienols. The most biologically active form is alpha-tocopherol, the most abundant in the diet being gamma-tocopherol. These molecules are present in large quantities in vegetable oils. They act essentially as antioxidants against reactive oxygen derivatives produced notably by the oxidation of fatty acids.

Advantageously, the composition according to the invention comprises:
spirulina or spirulina extract;
an extract of pollen of the species Secale cereale L.;
an extract of pollen of the species Zea mays L.;
an extract of pollen of the species Pinus sylvestris L.; and
an extract of pollen of the species Dactylis glomerata L.

Particularly advantageously, the composition according to the invention comprises:
spirulina or spirulina extract;
an extract of pollen of the species Secale cereale L.;
an extract of pollen of the species Zea mays L.;
an extract of pollen of the species Pinus sylvestris L.;
an extract of pollen of the species Dactylis glomerata L.; and
an extract of pistil of the species Zea mays L.

Alternatively, the composition according to the invention comprises:
spirulina or spirulina extract
an extract of pollen of the species Secale cereale L.;
an extract of pollen of the species Zea mays L.;
an extract of pollen of the species Pinus sylvestris L.;
an extract of pollen of the species Dactylis glomerata L.; and
selenium.

Also alternatively, the composition according to the invention comprises:
spirulina or spirulina extract;
an extract of pollen of the species Secale cereale L.;
an extract of pollen of the species Zea mays L.;
an extract of pollen of the species Pinus sylvestris L.;
an extract of pollen of the species Dactylis glomerata L.; and
at least one vitamin selected from D3, B6 and/or E.

Particularly advantageously, the composition according to the invention comprises:
spirulina or spirulina extract;
an extract of pollen of the species Secale cereale L.;
an extract of pollen of the species Zea mays L.;
an extract of pollen of the species Pinus sylvestris L.;
an extract of pollen of the species Dactylis glomerata L.;
an extract of pistil of the species Zea mays L.;
selenium; and
at least one vitamin selected from D3, B6 and/or E.

Even more advantageously, the composition according to the invention comprises:
spirulina or spirulina extract;
a cytoplasmic pollen extract from the species Secale cereale L.;
a cytoplasmic pollen extract from the species Zea mays L.;
a cytoplasmic pollen extract from the species Pinus sylvestris L.;
a cytoplasmic pollen extract from the species Dactylis glomerata L.;
a cytoplasmic pistil extract from the species Zea mays L.;
selenium; and
at least one vitamin selected from D3, B6 and/or E.

Even more advantageously, the composition according to the invention comprises:
spirulina or spirulina extract
an aqueous extract of pollen cytoplasm from the species Secale cereale L.;
an aqueous extract of pollen cytoplasm from the species Zea mays L.;
an aqueous extract of pollen cytoplasm from the species Pinus sylvestris L.;
an aqueous extract of pollen cytoplasm from the species Dactylis glomerata L.;
an aqueous extract of pistil cytoplasm from the species Zea mays L.;
selenium; and
at least one vitamin selected from D3, B6 and/or E.

In a preferred embodiment, the composition according to the invention comprises:
spirulina or spirulina extract;
an aqueous extract of pollen cytoplasm from the species Secale cereale L.;
an aqueous extract of pollen cytoplasm from the species Zea mays L.;
an aqueous extract of pollen cytoplasm from the species Pinus sylvestris L.;
an aqueous extract of pollen cytoplasm from the species Dactylis glomerata L.;
an aqueous extract of pistil cytoplasm from the species Zea mays L.;
selenium;
vitamin D3;
vitamin B6; and
vitamin E.

According to a particular embodiment, the composition according to the invention consists of:
spirulina or spirulina extract;
an aqueous extract of pollen cytoplasm from the species Secale cereale L.;
an aqueous extract of pollen cytoplasm from the species Zea mays L.;
an aqueous extract of pollen cytoplasm from the species Pinus sylvestris L.;
an aqueous extract of pollen cytoplasm from the species Dactylis glomerata L.;
an aqueous extract of pistil cytoplasm from the species Zea mays L.;
selenium;
vitamin D3;
vitamin B6; and
vitamin E.

According to a particular embodiment, the composition according to the invention consists of:
spirulina or spirulina extract;
60% to 95% an aqueous extract of pollen cytoplasm of the species Secale cereale L., preferably 60% to 86.5%, by weight of the total weight of the final pollen cytoplasm extract
4% to 20% an aqueous extract of pollen cytoplasm of the species Zea mays L., preferably 4% to 20%, by weight of the total weight of the final pollen cytoplasm extract;
0.5% to 3% an aqueous extract of pollen cytoplasm of the species Pinus sylvestris L. by weight of the total weight of the final pollen cytoplasm extract;
5% to 15% an aqueous extract of pollen cytoplasm of the species Dactylis glomerata L. by weight of the total weight of the final pollen extract
1% to 10% an aqueous extract of pistil cytoplasm of the species Zea mays L. by weight of the total weight of the final pollen cytoplasm extract;
selenium;
vitamin D3;
vitamin B6; and
vitamin E.

Preferentially, the composition used according to the invention comprises, excluding encapsulation or coating means:
30% to 50% spirulina or spirulina extract by weight of the total weight of the composition, preferentially about 36% by weight of the total weight of the composition;
20% to 50% an aqueous extract of pollen cytoplasm selected from the species Secale cereale L., the species Zea mays L., the species Pinus sylvestris L., and/or the species Dactylis glomerata L. by weight of the total weight of the composition; preferentially about 24.7% by weight of the total weight of the composition;
optionally 4% to 20% aqueous extract of pollen and pistil cytoplasm of the species Zea mays L. by weight of the total weight of the composition; preferentially 4.5% by weight of the total weight of the composition;
optionally 0.01% to 5% selenium by weight of the total weight of the composition; preferentially 4.5% by weight of the total weight of the composition;
optionally 0.001% to 5% vitamin D3 by weight of the total weight of the composition; preferentially 0.45% by weight of the total weight of the composition;
optionally 0.001% to 4% vitamin B6 by weight of the total weight of the composition; preferentially 0.39% by weight of the total weight of the composition; and
optionally 0.01% to 5% vitamin E by weight of the total weight of the composition, preferentially 4.0% by weight of the total weight of the composition;

Preferably, the daily dose of aqueous extracts of pollen and/or pistil of the composition according to the present invention is between 50 mg and 250 mg, more preferentially between 100 and 200 mg, even more preferentially between 110 and 150 mg. This daily dose is preferentially administered in 1, 2 or 3 doses (morning, midday and/or evening), for example in the form of 1, 2, 3, 4 or 6 gelcaps.

The final weight of the capsule is preferentially between 300 mg and 1000 mg. Even more preferentially, it is between 375 mg and 700 mg.

Particularly advantageously, the final weight of the capsule is 689 mg.

The compositions according to the invention are advantageously administered for a period of at least 10 days as a cure or three months, preferably six months. Advantageously, such a mode of administration will enable optimal modulation of immunity.

The invention also covers compositions as described above, for use as a medicament or dietary supplement.

In particular, the Applicant was able to demonstrate that the compositions according to the invention were particularly suitable for use as immunomodulators.

Thus, the invention also relates to oral compositions comprising spirulina or a spirulina extract and pollen obtained from plants belonging to the Secale, Zea, Pinus and/or Dactylis genera, for use as an immunomodulator.

An immunomodulator is a product that modifies immune responses.

This modification of the body's immune system can be caused by agents that activate or suppress its function.

The term immunomodulation according to the invention encompasses all therapeutic interventions aimed at modifying the immune response. Increasing the immune response is particularly desirable for preventing infections in immunodeficiency states, fighting established infections and combating cancer.

In cases of allergy, autoimmunity and organ transplantation, the aim is to weaken the immune response. Some allergies can be treated by specific desensitization. In cases of autoimmunity and transplantation, drugs are often used to attenuate all immune responses.

Immunity refers to the body's ability to defend itself against foreign substances, such as pathogenic microorganisms or cancer cells, by triggering an immune response.

When a foreign body enters the body, the immune system is triggered, producing messengers such as interleukins and cytokines.

As can be seen from the examples, the Applicant has been able to demonstrate that a synergy between spirulina and pollen extract, preferably selected from the species Secale cereale L. (rye), Zea mays L. (corn), Pinus sylvestris L. (pine), Dactylis glomerata L. (orchard grass), or a mixture thereof; has been identified in an in vitro test. In fact, this combination results in a strong release of cytokines (IL-1 beta, TNF alpha, IL-6, MCP-1, IL-8 and PGE-2) and thus a boost to the immune system.

Preferably, compositions according to the invention are used for the treatment and/or prevention of bacterial, fungal and/or viral infections.

According to the invention, the term “treatment” means an improvement, prophylaxis or reversal of a disease or disorder, or of at least one discernible symptom thereof. This is also an improvement, prophylaxis or reversal of at least one measurable physical parameter related to the treated disease or disorder, which is not necessarily perceptible to the subject. In another embodiment, the term “treatment” refers to inhibiting or slowing down the progression of a disease or disorder, either physically, for example, stabilizing a discernible symptom, physiologically, for example, stabilizing a physical parameter, or both. The term “treatment” also indicates the delay in the appearance of a disease or disorder. In certain particular embodiments of the invention, the compositions of interest are administered as a preventive measure. In this context, the term “prevention” refers to a reduction in the risk of acquiring a specified disease or disorder.

In particular, the Applicant has been able to demonstrate that the compositions according to the invention are particularly effective for the treatment and/or prevention of viral infections such as H1N1 influenza or SARS infections, and all their variants such as SARS-Cov-2/COVID-19/Coronavirus.

Influenza A (H1N1)v 2009 is an acute contagious respiratory illness caused by an influenza A virus subtype H1N1. This new influenza virus contains genes from several known viruses of swine, avian and human origin. It is different from the A (H1N1) virus responsible for seasonal influenza, which is of human origin. There are a large number of variants of influenza A, including: H1N1, H1N2, H1N8, H2N2, H2N3, H3N1, H3N2, H3N3, H3N8, H4N1, H5N1, H5N2, H5N3, H5N6, H5N8, H5N9, H7, H7N1, H7N2, H7N3, H7N7, H7N9, H9N2, H10N7, H10N8, H11N2, H11N9, H17N10, H18N11.

SARS-CoV-2 is a virus of the type known as a betacoronavirus. It is a positive-strand RNA virus with the largest genome of all RNA viruses. It has a spherical shape that is pleiomorphic, with its morphology varying according to the environmental conditions wherein it develops. The genome of this virus codes for four structural proteins required for the formation of a complete viral particle: protein S (Spike), protein M (Membrane), protein N (Nucleocapsid) and protein E (Envelope).

SARS-CoV-2 can cause a variety of symptoms, from fever and cough to respiratory distress (Respiratory Distress Syndrome—RDS), renal failure and even death in infected individuals.

The spike protein, also known as S protein, is a viral protein present on the surface of SARS-CoV-2. This protein can infect human respiratory epithelial cells via interaction between the Spike protein and the human ACE2 protein. The spike is a glycoprotein produced in large quantities, covering the surface of the virus and enabling it to penetrate human cells. The spike protein is a trimeric transmembrane protein of 180-200 kDa. It has an extracellular N-terminal domain and a small intracellular C-terminal domain. With a length of 1273 amino acids, it is composed of two subunits, 51 and S2. The 51 subunit includes a receptor-binding domain (RBD), which enables recognition of and binding to ACE2, a host cell surface receptor. The S2 subunit is the “stem” of the structure, and contains other basic elements required for membrane fusion. It also comprises a transmembrane domain, a fusion peptide, two sequences termed HR1 and HR2 (Heptapeptide Repeat sequences) and a cytoplasmic domain. Thanks to its two subunits, the spike protein mediates receptor binding and membrane fusion. When the RBD domain binds to ACE2, the S2 subunit undergoes a conformational change, anchoring the fusion peptide to the host cell. The HR1 and HR2 domains of the trimer form the 6-HB (six-Helical Bundle) through multiple interactions, bringing the viral membrane closer to that of the host and culminating in membrane fusion. Angiotensin-converting enzyme 2 (ACE2) is a cellular receptor homologous to angiotensin-converting enzyme (ACE). This receptor is expressed in the alveolar epithelial cells of the lungs, heart and kidneys. Through its involvement in the renin/angiotensin/aldosterone system, it plays a key role in cardiovascular and renal functions. ACE and ACE 2 have antagonistic effects. More specifically, activation of the ACE2 receptor is involved in vasodilation mechanisms, while ACE leads to vasoconstriction. Thus, the balance between activation and inactivation of these two receptors is essential for maintaining vascular homeostasis. In its physiological state, ACE2 therefore combats excessive vasoconstriction of the body's vessels. During infection with SARS-CoV-2, the virus binds to the ACE2 protein, enabling it to infect host lung cells, particularly type II pneumocytes. The balance between the effects mediated by ACE and ACE 2 proteins is then shifted in favor of those mediated by ACE, characterized by an increase in vasoconstriction, responsible for a wide variety of dysfunctions in patients infected with SARS-CoV-2. In fact, this abnormality of the renin/angiotensin system is linked to various cardiopulmonary pathologies, such as cytokinic shock responsible for acute inflammation of the lungs. What's more, in hypertensive patients with pre-existing obesity or diabetes, the imbalance accentuated by SARS-CoV-2 infection seems to lead to the development of the most severe forms, creating additional lesions (pulmonary fibrosis, cardiovascular lesions, etc.).

The aim, of course, is to prevent interaction between spike and ACE2 by antibodies, produced by the body when the immune system is activated, thus neutralizing the spike protein.

SARS-Cov-2 has a multitude of variants: “Nigerian” B.1.1.207, “British” VOC-202012/01, “Danish”, “South African” 501.V2, “Brazilian” or “Japanese” B.1.1.248, “Spanish” strain 20A EU.1, “California” CAL.20C, “New York” B.1.526.

According to a particular embodiment, the invention also relates to compositions as described above used for the treatment and/or prevention of immune deficiency, immunodeficiency (IMD) or immunodepression, which is a pathological situation linked to the insufficiency of one or more immunological functions.

A further object of the present invention relates to a food supplement comprising the composition as described above and used according to the invention.

Indeed, pollens or pollen preparations are widely consumed as a food supplement. They can be taken in their elementary form, which are pollens as such, and as powder in free or encapsulated form.

The composition according to the invention also comprises a physiologically acceptable medium proportioned to a reasonable benefit/risk ratio, comprising known excipients commonly used in phytotherapy such as binders, disintegrating agents, bulking agents, dispersing agents, agglomerating agents, lubricants, wetting agents, surfactants, emulsifiers, thickeners, flow agents, flavoring agents, sweetening agents, colorants, film-forming agents, stabilizers and/or preservatives.

The person skilled in the art will take care to choose these possible excipients and their quantity in such a way that they do not impair the attractive properties of the compositions used according to the invention.

Examples of excipients include cellulose, preferentially microcrystalline cellulose, and silicon dioxide.

In general, compositions according to the invention can be obtained by mixing different ingredients (in particular pollen and pistils) obtained separately by extraction processes, which may be identical or different. For example, the composition according to the invention may comprise spirulina, a first pollen and pistil extract combined with a second pollen extract. Advantageously, the first pollen and pistil extract is a combined extract of purified pollen and pistil cytoplasm, containing high activity of the antioxidant enzyme known as superoxide dismutase (SOD). The second pollen extract is a purified cytoplasmic extract of pure pollen. Pollen and pistils are selected and harvested separately and in a standardized way from members of the Poaceae family. The defined species are grown and harvested in separate fields under duly established quality control in accordance with the Good Agricultural Practices applicable to medicinal plants. The pollen is selected for the preparation of the second pollen extract, while the selected pollen and pistils are mixed in a standardized manner to produce the first extract formulation. During the extraction process, carried out in accordance with Good Manufacturing Practices, pollen and pistils are treated with enzymes to achieve the germination opening. The extract is then recovered by filtration, leaving out the pollen grain husks which can be allergenic. The extract is defined by high-performance liquid chromatography (HPLC) and gas chromatography (GC) to guarantee the quantity of active ingredients. The extract is mixed into a standardized formula. This standardization procedure results, for example, in tablets containing, in addition to spirulina, 100-140 mg of the first pollen and pistil extract and 20-60 mg of the second pollen extract. Preferentially, the amount of pollen and pistil extract in the final composition is 136 mg.

The invention thus also relates to a method for preparing a pollen and pistil extract from plant(s) preferentially belonging to the Pinaceae and/or Poaceae family comprising the following steps of:
a. aqueous extraction of pollen cytoplasm and optionally pistil cytoplasm;
b. spray-drying the aqueous extract(s) thus obtained;
c. recovering said aqueous extracts and mixing with spirulina or spirulina extract and optionally selenium and/or vitamins; and
d. recovering the resulting composition.

The Applicant has been able to demonstrate that the step of aqueous extraction of cytoplasm was particularly sensitive. Thus, the extraction temperature must be strictly less than 45° C. Beyond this temperature, one or more pollens and pistils detailed above are no longer present (no specific markers). The quality of the composition and/or its effectiveness are therefore not ensured.

Preferentially, the extraction temperature must be less than 42° C.

The length of the extraction step, for each of the extracts, is preferentially at least 6 hours, preferentially at least 10 hours, more preferentially still at least 12 hours.

In addition, the Applicant has been able to demonstrate that separation under conditions that are too sustained degraded the extract or extracts. Thus, when the extraction is coupled to a separation, the latter must not exceed 6000 revolutions per minute (rpm), preferentially 4500 rpm, more preferentially still 2800 rpm.

The method thus developed makes it possible to guarantee good traceability of the pollens and optionally pistils used. Indeed, the Applicant has been able to demonstrate that the extracts manufactured according to the methods of the prior art do not make it possible to find the presence of one or more markers of pollens and pistils and therefore, de facto, of the presence of the set of pollen and/or pistil extracts in the composition.

The compositions of the prior art are therefore not homogeneous and do not contain, in the final composition, all of the expected pollen and/or pistil extracts.

The method according to the invention advantageously comprises additional separation, filtration and/or evaporation steps making it possible to increase the concentration of the final extract or to optimize the preparation of said extract.

The method that is the subject of the invention also has the following advantages:
it allows standardization;
it has excellent reproducibility;
it allows a reduction in costs by optimizing and limiting the number of operations of the industrial tool.

It also makes it possible to be able to track the presence of the extracts in the composition according to the invention and thus to ensure its quality and consequently its effectiveness.

Among the advantages that make it possible to reduce handling and costs, these may be mentioned:
simplified planning;
optimized equipment occupancy;
reduced unproductive times (cleaning, changing batch, number of validations, etc.);
fewer reference numbers to manage;
fewer risky operations (cleaning, weighing, work procedures, costs reduced in validation, stability, formulation of the simplified tablet, microbiological risk, traceability, etc.).

The Applicant has demonstrated that the production of a composition from plant extracts often requires specific production steps, such as spray-drying and granulation. These steps can have an impact on the final composition and its constituents, such as proteins. Certain proteins which are present in the raw plant material are likely, due to the specific nature of the manufacturing process (typically spray drying), to lose their activity or to be lost. The Applicant has particularly been able to show that the use of an optimized spray drying process (temperature difference of 50° C. between the inlet temperature and the outlet temperature) avoided the denaturation of target proteins involved. The Applicant has also found that the presence in the final composition of at least one of these proteins or peptides derived from these target proteins helps to ensure the quality of the composition. The extracts used in the composition are thus optimal and pharmaceutical-grade, as the activity of the composition is not altered. Furthermore, by providing adequate quality control, better uniformity of the content may be ensured, which is important when patients are treated for specific symptoms and/or discomfort.

In addition, the composition is highly reproducible between different batches (in terms of quality and ingredients present), which allows quality control, standardization, traceability of batches and profiling of reproducible proteins. It thus complies with the good manufacturing practices for pharmaceutical products and food supplements on the market (pharmaceutical grade botanical products). This reproducibility also ensures that each new batch of pollen and/or pistil extract satisfies the established specifications and consequently at the same physiological activity as the batches used for clinical studies. The manufacturing method makes it possible to obtain the desired beneficial compound.

The present invention also relates to oral compositions which comprise at least spirulina and a component derived from pollen extract and/or pistil extract of plant(s) belonging to the Pinaceae and/or Poaceae family and in which the presence of one or more protein markers is detectable, whereby these protein markers are a good indication of the quality of the final composition. These markers can also be used for the standardization of the compositions.

Said markers or tracers are advantageously proteins or protein-derived peptides, said proteins being selected from the group of reticuline oxidase, endochitinase A, beta-1,3-glucanase, exopolygalacturonase, non-specific lipid transfer protein or any combination thereof.

According to the invention, the presence of specific markers of proteins or peptides originating from these proteins (markers) can be confirmed in the final composition, said markers being indicative of the quality of the composition and can therefore be used for the standardization and the quality control.

Indeed, the Applicant has been able to demonstrate that the presence of certain tracers or markers (protein or peptide derived from said protein) makes it possible to confirm the presence of certain pollen and/or pistil extracts in the final composition.

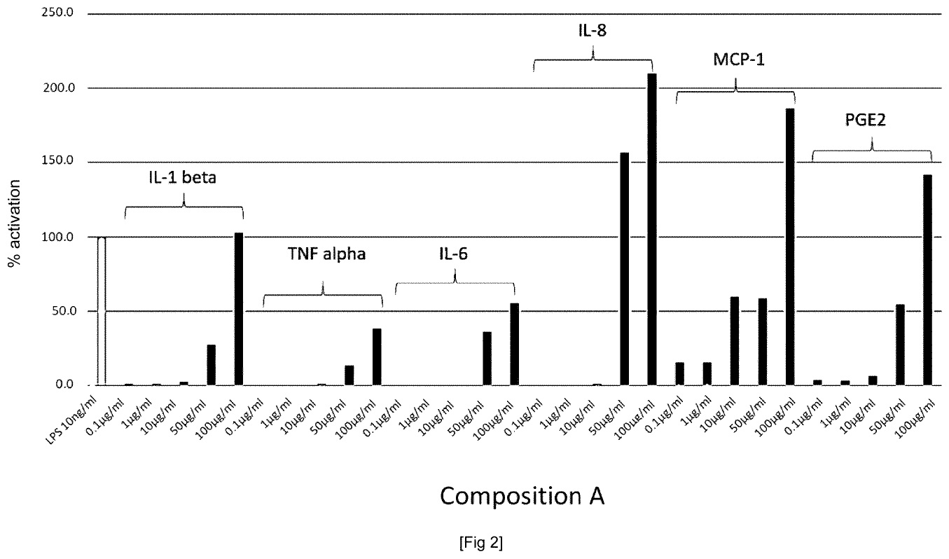

As seen in FIG. 2, the Applicant has been able to demonstrate that the synergistic effect of pollen cytoplasm extract and spirulina is dose-dependent. The higher the dose of composition A or B, the greater the synergistic effect and the better the cytokine activation.

The present invention will now be shown by means of the following examples:

### Example 1: Composition According to the Invention

### Composition A:

Dosage form: Capsule
Capsule weight: approx. 689 mg all-in, that is 550 mg without outer shell;

The percentages below are given by weight out of the total weight of the composition excluding encapsulation or coating means:
Spirulina: approximately 36% by weight of the total weight of the composition;
Aqueous pollen extract, here from pollen cytoplasm: approx 25% by weight of the total weight of the composition;
Excipients: approximately 36% by weight of the total weight of the composition.

TABLE 1
Aqueous pollen extract ingredients expressed by weight of the
total weight of the final pollen extract (pollen cytoplasm):

| Ingredients | % Quantity |
|---|---|
| Secale cereale L. Pollen | 60-95, preferably 60-90.5 |
| Zea mays L. Pollen | 4-20 |
| Dactylis glomerata L. Pollen | 5-15 |
| Pinus sylvestris L. Pollen | 0.5-3 |

Other ingredients in the final product may include microcrystalline cellulose, potato starch and magnesium stearate. The plant-based capsule is based on HPMC: hydroxypropylmethylcellulose. The recommended daily dose is 1 capsule per day, to be taken in the morning or evening.

The pollen extract (pollen cytoplasm) in the above capsule was obtained using a conventional process for preparing pollen and/or pistil extracts.

### Example 2: Composition According to the Invention

### Composition B

Dosage form: Capsule
Capsule weight: approx. 689 mg all-in, 550 mg without outer shell.

The percentages below are given by weight out of the total weight of the composition excluding encapsulation or coating means: Spirulina: approximately 36% by weight of the total weight of the composition;
Aqueous pollen extract, here from pollen cytoplasm: approx 25% by weight of the total weight of the composition;
Selenium: approximately 4.5% by weight of the total weight of the composition;
Vitamin D: approximately 0.5% by weight of the total weight of the composition;
Vitamin B6: approximately 0.4% by weight of the total weight of the composition;
Vitamin E: approximately 4.0% by weight of the total weight of the composition;
Excipients: approximately 29.4% by weight of the total weight of the composition.

TABLE 2
Extract ingredients expressed by weight of the
total weight of the final pollen extract:

| Ingredients | % Quantity |
|---|---|
| Secale cereale L. Pollen | 60-95, preferably 60-88.5 |
| Zea mays L. Pollen | 5-20, preferably 4-20 |
| Dactylis glomerata L. Pollen | 5-15 |
| Pinus sylvestris L. Pollen | 0.5-3 |
| Zea mays L. pollen and pistil | 1-10 |

Other ingredients in the final product may include microcrystalline cellulose, potato starch and magnesium stearate. The plant-based capsule is based on HPMC: hydroxypropylmethylcellulose. The recommended daily dose is 1 gelcap per day, to be taken in the morning or evening.

The pollen extract (pollen cytoplasm) in the above capsule was obtained using a conventional process for preparing pollen and/or pistil extracts.

### Example 3: Procedure Used to Generate Results for Studying the Effect of Composition A on Cytokine Production

Monocyte cell cultures: Primary human monocytes were prepared from the buffy coat of healthy human blood donors using a standardized procedure.

Monocyte cell processing and ALAMARBLUE® test: Cells were seeded in 96-well plates at a density of 220000 cells/well for viability measurements. The test elements were dissolved in cell culture media. 1 μL of stock solution or dilutions were added per well (100 μL). Monocytes were seeded in 96 wells and incubated with: NaF (250 μg/mL), a positive control, a medium control and increasing concentrations of the elements to be tested. After 24 hours incubation, 10 μL ALAMARBLUE® (Biosource, USA) was added to each well. After 2 hours, fluorescence was measured with a fluorescence spectrophotometer using filter settings 544EX nm/590EM nm. The amount of fluorescence is proportional to the number of living cells, and corresponds to the metabolic activity of the cells. Damaged, non-viable cells have lower innate metabolic activity and therefore generate a proportionally lower signal than healthy cells. The active ingredient in ALAMARBLUE® (resazurin) is a cell-permeable, non-toxic, blue-colored, virtually non-fluorescent compound. When it penetrates cells, resazurin is reduced to resorufin, which produces a very bright red fluorescence. Viable cells continuously convert resazurin to resorufin, generating a quantitative measure of viability and cytotoxicity.

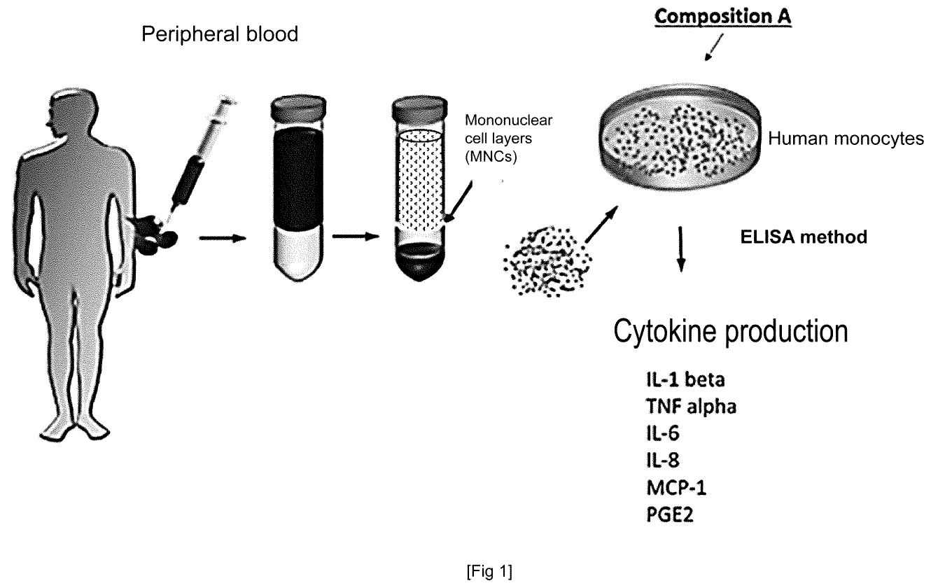

Immunomodulation in human monocytes: As shown in FIG. 1, primary human monocytes were isolated from the buffy coat of three healthy human blood donors. Cells were seeded in 24-well plates at a density of 2200000 cells/well for ELISA measurements. Monocytes were incubated with test elements (5 different concentrations) for 24 h. LPS (10 ng/mL, Salmonella typhimurium SL1181) served as a positive control for cytokine release. After 24 h, supernatants were removed, centrifuged and studied for IL-1β, MCP-1, IL-6, TNF and IL-8 concentrations by ELISA using the manufacturer's protocol (n=6, 3 different donors).

The tested concentrations of the individual ingredients refer to the concentrations in composition B, that is.:
200 mg Spirulina (36%)
136 mg pollen extract (25%)
25 mg Selenium (4.5%)
2.5 mg Vitamin D3 (0.5%)
2.15 mg Vitamin D6 (0.4%).

### Example 4: Effect of Composition A According to the Invention on Cytokine Production

As can be seen from FIG. 2, administering Composition A induces the release of IL-1β, TNF-α, IL-8, MCP-1 and PGE2 in human monocytes, starting from a concentration of 0 μg/mL being maximal at the tested dose of 100 μg/mL.

Induction is dependent on the dose of the composition.

IL-1β is an interleukin secreted by macrophages, and plays a pro-inflammatory role. IL-111 release was induced from 10 μg/mL up to 100 μg/mL. Between 50 μg/mL and 100 μg/mL, the activation percentage rises from 25% to 100%.

TNF-α (Tumor Necrosis Factor) is a cytokine involved in inflammation. TNF-α release was induced from 50 μg/mL up to 100 μg/mL. Between 50 μg/mL and 100 μg/mL, the activation percentage rises from 20% to 40%.

IL-6 is an interleukin secreted by macrophages, T lymphocytes and endothelial cells. It enables the synthesis of acute-phase proteins and the proliferation of antibody-producing cells. IL-6 release was induced from 50 μg/mL up to 100 μg/mL. Between 50 μg/mL and 100 μg/mL, the activation percentage rises from 40% to 55%.

IL-8 is an interleukin produced by epithelial cells following the detection of potentially pathogenic microbiological or chemical agents. IL-8 release was induced from 50 μg/mL up to 100 μg/mL. Between 50 μg/mL and 100 μg/mL, the activation percentage rises from 150% to 210%.

MCP-1 (Monocyte Chemoattractant protein 1) is a chemokine-type cytokine, and is therefore attractive to certain immune cells. MCP-1 release was induced from 0.1 μg/mL up to 100 μg/mL. Between 50 μg/mL and 100 μg/mL, the activation percentage rises from 60% to 190%.

PGE2 (Prostaglandin E2) is a fatty acid of the prostaglandin class, playing an important role in inflammation. PGE-2 release was induced from 0.1 μg/mL up to 100 μg/mL. Between 50 μg/mL and 100 μg/mL, the activation percentage rises from 60% to 145%.

Composition A can therefore be said to induce the production of several pro-inflammatory and immune cell-attracting cytokines. It has the ability to activate and boost the immune system.

Such results, not shown, have also been observed using composition B as described in example 2.

### Example 5: Synergy Between Pollen Extracts (Pollen Cytoplasm) and Spirulina

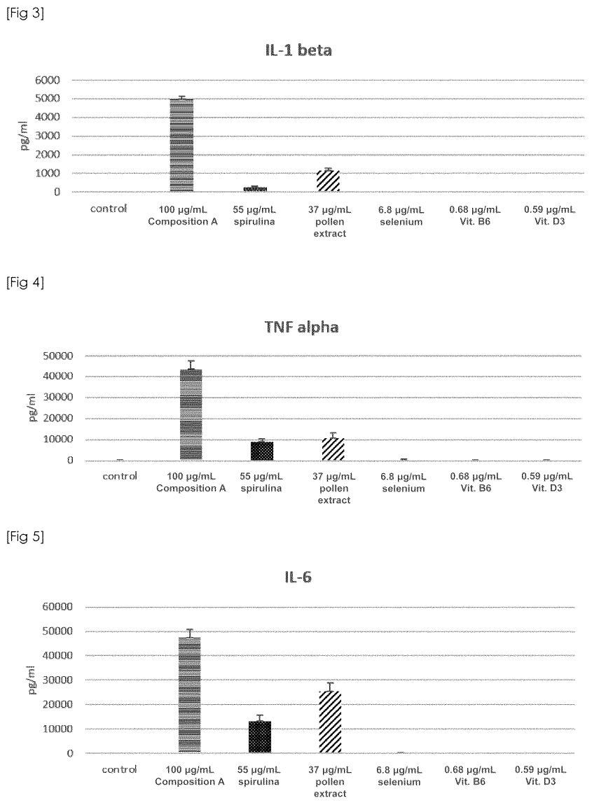

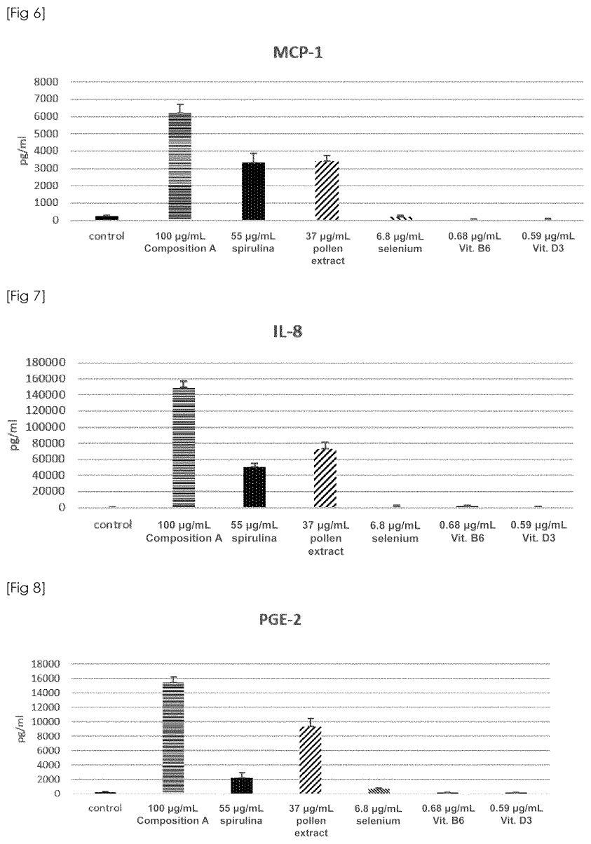

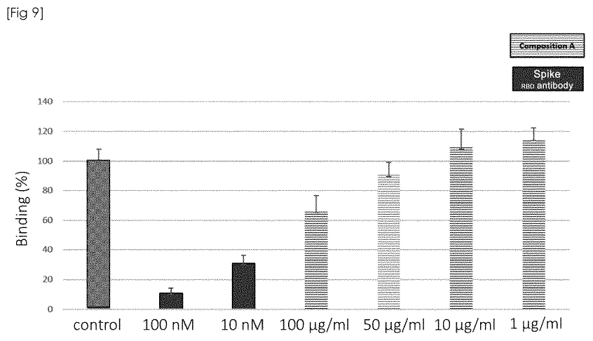

The effect of composition A is greater than the effects of the individual ingredients spirulina and pollen extract (pollen cytoplasm):
IL-1β: as shown in FIG. 3, spirulina (55 μg/mL) induces a release of 200 μg/mL IL-1β. Pollen extract (37 μg/mL) induces a release of 1,000 μg/mL IL-1β. Composition A (100 μg/mL) induces a release of 5000 μg/mL IL-1β.
TNF-α: as shown in FIG. 4, spirulina (55 μg/mL) induces a release of 10,000 μg/mL TNF-α. Pollen extract (37 μg/mL) induces a release of 10,000 μg/mL TNF-α. Composition A (100 μg/mL) induces a release of 45,000 μg/mL TNF-α.
IL-6: as shown in FIG. 5, spirulina (55 μg/mL) induces a release of 12,000 μg/mL IL-6. Pollen extract (37 μg/mL) induces a release of 25,000 μg/mL IL-6. Composition A (100 μg/mL) induces a release of 47,000 μg/mL IL-6.
MCP-1: as shown in FIG. 6, spirulina (55 μg/mL) induces a release of 3,100 μg/mL MCP-1. Pollen extract (37 μg/mL) induces a release of 3,200 μg/mL MCP-1. Composition A (100 μg/mL) induces a release of 6,000 μg/mL MCP-1.
IL-8: as shown in FIG. 7, spirulina (55 μg/mL) induces a release of 15,000 μg/mL IL-8. Pollen extract (37 μg/mL) induces a release of 25,000 μg/mL IL-8. Composition A (100 μg/mL) induces a release of 47,000 μg/mL IL-8.
PGE-2: as shown in FIG. 8, spirulina (55 μg/mL) induces a release of 2,000 μg/mL PGE-2. Pollen extract (37 μg/mL) induces a release of 9,500 μg/mL PGE-2. Composition A (100 μg/mL) induces a release of 15,500 μg/mL PGE-2.

Arguably, the effects of composition A are even stronger than the sum of the effects of spirulina and pollen extract (pollen cytoplasm), indicating possible synergistic activities of the ingredients with regard to immunomodulation. Neither selenium, vitamin B6 nor vitamin D3 had any effect on basal cytokine release in human monocytes up to the highest concentration studied (0.68 μg/mL and 0.59 μg/mL respectively).

Such results, not shown, have also been observed using composition B as described in example 2.

### Example 6: Examination of the Effects of Composition A on ACE2 Interaction: SARS-CoV-2

Method: Composition A was prepared in 5×PBS/DMSO stock solutions (final conc. DMSO 0.1%). ACE2 interaction: SARS-CoV-2 Spike (RBD) was tested according to the instructions of the supplier (BPS Bioscience, USA). As a positive control, the Spike S1-neutralizing antibody (BPS Bioscience 100793) was included in the test (50 nM and 100 nM). After incubation, chemiluminescence was immediately read (luminescence measurement time: 0.1 s, Victor X5, Perkin Elmer, USA).

TABLE 3
Results of ACE2 inhibitor screening test: Spike
S1 and the inhibitory effect of composition A.

| | | | ACE2: SARS-CoV-2 Spike | |
|---|---|---|---|---|
| | | | (RBD) | |
| | | | | Standard |
| | Concentration | | Binding (%) | deviation (%) |
| Control | — | | 100 | 8.0 |
| Anti-Spike S1 | 100 | nM | 10.8 | 3.3 |
| antibodies | 10 | nM | 30.9 | 5.3 |
| Composition A | 100 | μg/mL | 66.3 | 10.4 |
| | 50 | μg/mL | 90.4 | 8.5 |
| | 10 | μg/mL | 109.1 | 12.5 |
| | 1 | μg/mL | 114.9 | 7.2 |

The data in the table represent the mean n=4±SD (the control link was set to 100%)

Table 3 above shows the percentage of binding between ACE2 and Spike as a function of the presence of anti-Spike antibodies and composition A.

The control shows that Spike and ACE2 do indeed bind at 100%±8.0%.

As seen in FIG. 9:
100 nM of anti-Spike antibodies inhibit Spike RBD binding to the ACE2 protein at 90%±3.3%.
10 nM of anti-Spike antibodies inhibit Spike RBD binding to the ACE2 protein at 70%±5.3%.

Composition A reduces Spike RBD binding to the ACE2 protein from 40%±10.4% at 100 μg/mL and 10%±8.5% at 50 μg/mL.

We can therefore conclude that composition A is effective and represents a good alternative for reducing Spike RBD binding to the ACE2 protein.

Such results, not shown, have also been observed using composition B as described in Example 2.

1. An oral composition comprising spirulina or a spirulina extract, characterized in that it further comprises an aqueous extract of pollen cytoplasm, said pollen(s) being obtained from plants belonging to the Secale, Zea, Pinus and/or Dactylis genera, and optionally:
extract of pistil(s);
selenium; and/or
vitamins.

2. The composition according to claim 1, characterized in that it further comprises a pistil extract which is preferably an aqueous extract of pistil cytoplasm obtained from the Zea plant.

3. The composition according to claim 1, characterized in that it further comprises selenium.

4. The composition according to claim 1, characterized in that it further comprises at least one vitamin, preferentially vitamin D3, B6 or E, taken alone or as a mixture.

5. The composition according to claim 1, characterized in that it comprises:
spirulina or spirulina extract;
an aqueous extract of pollen cytoplasm from the species Secale cereale L.;
an aqueous extract of pollen cytoplasm from the species Zea mays L.;
an aqueous extract of pollen cytoplasm from the species Pinus sylvestris L.;
an aqueous extract of pollen cytoplasm from the species Dactylis glomerata L.;
an aqueous extract of pistil cytoplasm from the species Zea mays L.;
selenium;
vitamin D3;
vitamin B6; and
vitamin E.

6. The composition according to claim 5, characterized in that it comprises:
spirulina or spirulina extract;
60% to 95% an aqueous extract of pollen cytoplasm of the species Secale cereale L., preferably 60% to 86.5%, by weight of the total weight of the final pollen extract;
4% to 20% an aqueous extract of pollen cytoplasm of the species Zea mays L., preferably 4% to 20%, by weight of the total weight of the final pollen cytoplasm extract;
0.5% to 3% an aqueous extract of pollen cytoplasm of the species Pinus sylvestris L. by weight of the total weight of the final pollen cytoplasm extract;
5% to 15% an aqueous extract of pollen cytoplasm of the species Dactylis glomerata L. by weight of the total weight of the final pollen cytoplasm extract;
1% to 10% an aqueous extract of pistil cytoplasm from the species Zea mays L. by weight of the total weight of the final pollen cytoplasm extract;
selenium;
vitamin D3;
vitamin B6; and
vitamin E.

7. The composition according to claim 1, characterized in that it comprises, excluding encapsulation or coating means:
30% to 50% spirulina or spirulina extract by weight of the total weight of the composition, preferentially about 36% by weight of the total weight of the composition;
20% to 50% an aqueous extract of pollen cytoplasm selected from the species Secale cereale L., the species Zea mays L., the species Pinus sylvestris L., and/or the species Dactylis glomerata L. by weight of the total weight of the composition; preferentially about 24.7% by weight of the total weight of the composition;
optionally 4% to 20% aqueous extract of pollen and pistil cytoplasm of the species Zea mays L. by weight of the total weight of the composition; preferentially 4.5% by weight of the total weight of the composition;
optionally 0.01% to 5% selenium by weight of the total weight of the composition; preferentially 4.5% by weight of the total weight of the composition;
optionally 0.001% to 5% vitamin D3 by weight of the total weight of the composition; preferentially 0.45% by weight of the total weight of the composition;
optionally 0.001% to 4% vitamin B6 by weight of the total weight of the composition; preferentially 0.39% by weight of the total weight of the composition; and
optionally 0.01% to 5% vitamin E by weight of the total weight of the composition; preferentially 4.0% by weight of the total weight of the composition.

8. The composition according to claim 1, for use as a medicament.

9. An oral composition comprising spirulina or a spirulina extract and an aqueous extract of pollen cytoplasm(s) obtained from plants belonging to the Secale, Zea, Pinus and/or Dactylis genera, for use as an immunomodulator.

10. The oral composition according to claim 1 for use as an immunomodulator.

11. The composition for use according to claim 8, characterized in that it is used for the treatment and/or prevention of bacterial, fungal and/or viral infections.

12. The composition for use according to claim 8, characterized in that it is used for the treatment and/or prevention of SARS-Cov-2/COVID-19/Coronavirus infection.

13. A method for preparing a composition according to claim 1, comprising the following steps of:
a) aqueous extraction of pollen cytoplasm and optionally pistil cytoplasm;
b) spray-drying the aqueous extract(s) thus obtained;
c) recovering said aqueous extracts and mixing with spirulina or spirulina extract and optionally:
selenium; and/or
vitamins; and
d) recovering the resulting composition.
